(19) United States
(12) Patent Application Publication
(10) Pub. No.: US 2003/0013084 A1
(43) Pub. Date: Jan. 16, 2003

(54) Organometallic probe

(75) Inventor: Gorm Danscher, Aarhus C (DK)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: Berlock ApS, Aarhus (DK)

(21) Appl. No.: 10/192,853

(22) Filed: Jul. 11, 2002

Related U.S. Application Data
(60) Provisional application No. 60/304,405, filed on Jul. 12, 2001.

(51) Int. Cl. C12Q 1/70; G01N 33/553

(52) U.S. Cl. 435/5; 436/525

(57) ABSTRACT

An organometallic probe comprising a colloidal core, for example a ,old colloid, to which a membrane translocating signal (MTS), for example an HIV-tat fragment, is directly attached. The organometallic probe is suited for silver enhancement techniques.

### FIELD OF THE INVENTION

The present invention relates to an organometallic probe for biological specimens comprising a colloidal core with a membrane translocating signal (MTS) molecule attached thereto. The invention further relates to a method for staining a biological entity, and to a method for identifying a biological entity in a surrounding substance.

### BACKGROUND OF THE INVENTION

A variety of methods have been developed for staining specific biological entities in order to trace these entities in a substance, for example an organism. It is of paramount importance to have techniques for tracing the fate of biological cells, bacteria, nuclei, or vesicles that have a special origin, have been treated in a special way, are auto-, allo-, or heterotransplanted or inoculated into cultures of the same or different species of micro-organisms.

A few methods have been developed for this kind of tracing. They are either based on the concept of separating marked cells/bacteria by destroying the cellular connections/morphology of the tissues and then separate the marked cells by magnetic separation, or they are based on location of the iron target cells in tissue sections by NMR imaging or immunofluorescence.

In 1981 it was found that tissues from organisms being exposed to gold salts, for example aurothiosulphate, contained gold ions bound to macromolecules and that these gold ions could be traced by autometallography (AMG) if first reduced to metallic gold clusters by for example UV light. This is described in the article by G. Danscher: “Localisation of gold in biological tissues. A photochemical method for light and electron microscopy.” published in Histochemistry 1981; 71: 81-88. This new knowledge of gold clusters being capable of igniting AMG silver enhancement was soon applied in tracing of molecules marked with colloidal gold particles. The use of colloidal gold particles had been introduced a few years before as a marker of, for example, antibodies to be traced in the electron microscope, which is references in the article by M. Horisberger and J. Rosset: “Colloidal gold, a useful marker for transmission and scanning electron microscopy,” which has been published in J. Histochem. Cytochem. 1977; 25: 295. The introduction of AMG for colloidal gold visualisation as published by G. Danscher and J. O. R. Norgaard: “Light microscopic visualisation of colloidal gold on resin-embedded tissue.” in J. Histochem. Cytochem. 1983; 31/12: 1394-1398; and by C. S. Holgate, P. Jackson, P. N. Cowen, and C. C. Bird: “Immunogold-silver staining: New method of immunostaining with enhanced sensitivity,” in J. Histochem. Cytochem. 1983; 31: 938-944, started a new era in several fields of research. In particular because of its use in hospital laboratories, it was subject to an intense commercial interest.

Soon, several companies produced both AMG silver enhancement kits, but also a huge variety of gold-marked antibodies and enzymes became available on the market. In order to increase the tissue penetrance of the colloidal gold-marked antibodies, it became an issue to make the colloidal gold particles as small as possible and to develop gold cluster containing molecules that could optimise the technique.

In U.S. Pat. No. 5,728,590, an organothiol metal cluster compound is disclosed for detecting the histological localisation of biological substances, for example specific molecules, lipids or proteins. The organometallic probe comprises a metal core surrounded by a polymer shell to which biological substances as proteins, peptides, antibodies, lipids, carbohydrates, nucleic acids, drugs or hormones are covalently attached to impart desirable physical and chemical properties to the probe. This metal cluster was introduced as an alternative to colloidal gold particles because of, among other things, its small size that allowed the tagged substances to penetrate a little further into embedded tissues. This metal cluster suffers from having a strongly reduced catalytic power for inducing the binding to silver. Therefore, it is less attractive for the silver enhancement techniques as far from all of the available probes are able to be silver enhanced in the final biological entity, for example a biopsy, to be investigated. The reason for this reduced catalytic power is believed to be the polymer coating which surrounds the metal core.

Membrane translocating signal peptides have been used for some time to internalise magnetic nanospheres or biotinylated polystyrene micro-spheres (FluoSpheres®) into membrane-limited compartments like cells or bacteria. For example, Lewin, N. Carlesso, X. W. Tang, D. Cory, D. T. Schradden and R. Weissleder have described this kind of cell tracing in their article “Tat peptide-derivatized magnetic nanoparticles allow in vivo tracking and recovery of progenitor cells”, published in Nat. Biotechnol. April 2000, 18(4); 401-414. Another description of this technique is found in “High-Efficiency Intracellular Magnetic Labeling With Novel Superparamagnetic-Tat Peptide Conjugates” by Lee Josephson, Ching-Hsuan Moore, Anna More and Ralph Weissleder published in Bioconjugate Chem. 1999, 10, 186-191. In these papers, the authors describe a technique for labelling cells by superparamagnetic nanoparticles for later detection by magnetic resonance (MR). In order to achieve a proper internalisation of the magnetic particles into the cells, the particles were bound to a new and for this purpose very effective HIV-Tat peptide. Detection by magnetic resonance suffers from low spatial resolution, which is also demonstrated by the MR images presented in the publications. This method is rather a scientific tool for demonstration of labelling principles than an applicable method for investigation at microscopic resolution and may only be used in connection with MR scanning of larger objects as organs with high levels of cells loaded with magnetic iron oxide particles. In the publications, it is shown that the internalised iron oxide particles can be visualised by fluorescence microscopy. However, this is only possible as long as the tat molecules attached to the dextran encased iron oxide particle exist. The tat peptide is rather fast metabolised leaving the iron oxide particle without possibility of being traced by immunohistochemical techniques. Therefore, the magnetic iron oxide technique is not suited for histological investigation. Also, it is known that iron oxide particles can be toxic.

### DESCRIPTION OF THE INVENTION

It is an object of the invention to provide an improved organometallic probe in order to ameliorate existing histochemical cell labelling techniques. In particular, it is the object of the invention to ensure tagging and tracing of biological compartments.

This object is achieved with an organometallic probe comprising a colloidal core with a membrane translocating signal (MTS) molecule attached thereto, wherein said core contains at least one from the group consisting of a metal, a metal selenide or a metal sulphide, for example gold, bismuth sulphide, bismuth selenide, mercury sulphide, mercury selenide, silver sulphide, silver selenide, copper sulphide, or copper selenide, or a combination thereof. Such a combination may for example be achieved by a mixture of these materials or by combining particles of different of these materials. Metal containment in this context has to be understood as a containment of the metallic form of the substance in the core in contrast to containment of a metal oxide, which is not meant by the term “metal containment”. As will be apparent from the following, a colloidal gold particle is the most preferred due to its non-toxicity in contrast to iron oxide, which may have toxic side effects.

Several MTS molecules have been described, for example MTS peptide derived from anti-DNA monoclonal antibody as described by A. Avrameas et al: “Polyreactive anti-DNA monoclonal antibodies and a derived peptide as vectors for the intracytoplasmic and intranuclear translocation of macromolecules.” as published in Proc. Natl.Acad.Sci. U.S.A. 95, 5601-6 (1998). Another MTS, namely VP22 herpes virus protein is described by Phelan et al. in the article: “Intercellular delivery of functional p53 by the herpes virus protein VP22.”, which is published in Nat. Biotechnol.16, 440-31998. Preferably in connection with the invention, a HIV-tat molecule is used. Related information for a HIV-1 tat peptide can be found in an article by Fawell et al.: “Tat-mediated delivery of heterologous proteins into cells,” published in Proc.Natl.Acad.Sci. U.S.A. 91, 664-8, (1994). The HIV-1 tat protein has been shown to freely travel through cellular and nuclear membranes as presented by A. D. Frankel and C. O. Pabo in their publication: “Cellular uptake of the tat protein from human immunodeficiency virus,” printed in Cell 55, 1189-93 (1988). Another example of a MTS molecule is the third helix of the homeodomain of Antennapedia.

It is an advantage to bind the MTS directly to the colloidal core in contrast to having the metal core completely surrounded by a polymer or other organic substances, because the core in this way has a high catalytic power for silver enhancement, which increases the tracing sensitivity dramatically. A tight binding of the MTS on gold colloids is an easy task because of the negative charges on the surface of the nanogold particle. Thus, no coating, for example with a polymer or with Dextran® is needed. Typical sizes for these gold colloids are between 0.4 nm and 200 nm, preferably between 1 nm and 40 nm.

An organometallic probe according to the invention may be used for staining a biological entity, for example a cell, a cell organelle, for example a nucleus, a mitochondrion, a lysosome, a vesicle, for example a secretory vesicle or a synaptic vesicle, a protozoon, a bacterium, or a fungi, into which the probe is internalised after which the probe is subjected to silver enhancement for visual inspection. Such tracing of the biological entity or its progeny may be performed in, for example, a solution, a cell culture, or an organism or part thereof. For example, the technique also opens up the possibility of tracing whether and if so, where in the organism inoculated cells/bacteria/fungi settle and how well they proliferate.

Special interest has this tracing in connection with organisms as plants, human beings or animals, where the animal for investigation may be sacrificed.

If the tracing takes place in humans the biological entities, for example lymphocytes, may be isolated by a biopsy and and probes with the MTS tags internalised before the entities, for example lymphocytes, are replace in the same person or a recipient person. Probes may also be bound to other entities as drugs or virus. After inoculation in an organism, the tagged drugs or virus can be traced to the internalised cells in tissues from the exposed, and eventually sacrificed, animal or in biopsies. In the microscope the tagged cells can be identified and the localization of them and their progeny be identified.

There are a number of advantages of using autometallographic (AMG) tags as compared to iron oxide cores or polystyrene spheres, where AMG tags in the following is to be understood as comprising a colloidal gold core according to the invention, though also cores made of bismuth sulphide, bismuth selenide, mercury sulphide, mercury selenide, silver sulphide, silver selenide, copper sulphide, or copper selenide may have comparable properties.

A first advantage is the fact that these AMG tags can be silver enhanced to visible dimensions even if present only in a small number, usually less than 10 AMG tags per location are sufficient to identify a tagged cell. In theory, just a single AMG tag should suffice.

Iron oxide is not able to catalyse autometallographic silver enhancement. As, normally, cells are loaded with hundreds of thousands of AMG-tags per cell, the obtained signal is impressively strong.

The second advantage is that the cells/bacteria and their progeny in many generations can be traced efficiently in tissue sections from biopsies/autopsies, in cell cultures, and in smears from excretes/ejaculates/blood samples and the like because of the above mentioned exceptional sensitivity. This is not possible with iron oxide particles that cannot be traced histologically after removal of the MTS molecule, which typically occurs rather quickly, typically within a few hours, in biological compartments due to degradation, as the MTS is a foreign body in the compartment and subject for chemical attack. Also, iron oxide may be decomposed and even removed as a result of chemical break down. In contrast, gold particles can be traced by AMG also after the attached MTS molecules has be removed, i.e. their presences in cell or bacteria, or their progeny, and can be visualized by AMG as the gold particles are the objects for silver enhancement. Gold cannot be oxidised in a cell and does not react chemically with the organelles or molecules in the cell, which explains why the particles remain unchanged and can be followed in generations of dividing cells.

A further advantage is the fact that AMG tags after silver enhancement can be traced at both light microscopic and ultrastructural levels, for example with electron microsopy.

Furthermore, the preparations can be stored for tens of years and reused for further or repeated investigations, a quality that the described technique using iron oxide particle do not posses nor the techniques with polystyrene spheres.

It is also important to stress that metallic gold has no known adverse effects to living cells or organisms, and the handling of the AMG-tag technique according to the present invention represents no known or even speculative danger to people or the environment.

The internalising tags may be delivered as solutions ready for exposure of the cells in question or as a solution for injection. In the following, examples are given for the preparation and for the staining technique.

An Example of How to Prepare AMG-tags: Colloidal gold particles can, for example, be prepared according to the method of G. Frens: “Controlled nucleation for the regulation of the particle size in monodisperse gold suspensions”, published in Nat. Phys. Sci. 1973; 241: 20. This procedure results in a colloidal solution composed of gold particles having an average size of 14.5 nm. Colloidal gold particles can be produced in sizes from 0.1-100 nm by many different techniques, which is well known and described in prior art.

An Example of How to Make an MTS Complex is Given in an Article by L. Josephson, C.-H. Tung, A. Moor, and R. Weissleder: “High-efficiency intracellular magnetic labelling with novel superparamagnetic-tat peptide conjugates. Bioconjugate Chem. 1999; 10: 186-191”. A peptide containing the translocating sequence of the tat peptide was synthesized on an automatic synthesizer (PS3, Rainin, Woburn, Mass.) by Fmoc chemistry using 2-(1H-benzotriazole-1-yl)-1,1,3,3-tetramethyluronium hexafluorophosphate (HBTU)/N-hydroxybenzotriazole (HOBt) as activating agent. The peptide is referred to as tat (FITC). The sequence is Gly-Gly-Cys-Gly-Arg-Lys-Lys-Arg-Arg-Gln-Arg-Arg-Arg-Lys-(FITC)-NH₂ (the italicised amino acids correspond to residues 48-57 of the tat protein). Fmoc-Lys(Dde) was anchored to 0.1 mmol of Rink amide MBHA resin® (NovaBiochem®, San Diego, Calif.) and followed with other amino acids, for example Fmoc-Arg(Pbf), Fmoc-Gln(Trt), Fmoc-Lys(Boc), Fmoc-Gly, and Fmoc-Cys(Trt). The N-terminal was finally capped with t-Boc-Gly. Thereafter, the Dde group on the C-terminal lysine residue was selectively removed with 10 mL of 2% hydrazine in DMF (2×3 min) and the deprotected amino group was reacted with 0.4 mmol of fluorescein isothiocyanate (FITC) (Aldrich, Milwaukee, Wis.) in 5 mL of DMSO/diisopropylethylamine (20% v/v) overnight. The peptide was cleaved by 5 mL of TFA/thioanisole/ethandithiol/anisole (90/5/3/2) and purified by C18 reversed-phase HPLC. MALDI-MS (M+H)⁺.

An Example of How to Bind MTS to the AMG-tag: The following procedure is performed in accordance with the procedure described by Bendayan: “Ultrastructural localisation of nucleic acids by the use of enzyme-gold complexes” as published in J. Histochem. Cytochem. 1981; 29: 531. Reference is also made to the article by G. Danscher and J. O. R. Norgaard: “Light microscopic visualization of colloidal gold on resin-embedded tissue”. J. Histochem. Cytochem. 1983; 31: 1394-1398. Ten milliliters of the gold suspension were adjusted to pH 9.0 with 0.2 M K₂CO₃ and 0.5 mg Rnase A (Sigma(®) dissolved in 0.1 ml H₂ O was added. At this pH, close to the isoelectric point of the protein, a stabilized gold sol was formed. After centrifugation at 25,000 rpm for 30 min the pellet was suspended in phosphate buffered saline (PBS) at pH 7.5.

Exposure of Isolated Cells/Bacteria to AMG-tag-MTS Complex: The isolated cells/bacteria obtained by the most appropriate technique for the cells/bacteria in question are placed in a suitable medium at 37° C. and the AMG-tag-MTS complex dissolved in a phosphate buffer is added. After an hour, the cells/bacteria are washed several times and are now ready for injection into embryos, organisms, organs or inoculated into cell or bacteria cultures.

Tracing the AMG-tag Loaded Cells/Bacteria in Tissue Sections/Cellular Samples and Cultures:

Biopsies or autopsies from fixed and unfixed organisms, for example embryos or experimental animals, are handled according to the procedure found to be most suited for the studies additionally performed on the final tissue sections. This includes postfixation and embedding in paraffin, Epon, or metachrylate before being sectioned or being frozen and sectioned on a cryostat. They can be either of immunohistochemical/immunocytological nature, morphological analyses, or other approaches. The AMG-tags present in the target cells/bacteria and their progeny do not demand special precautions of any kind as long as they are not treated with substances that dissolve the AMG-tag used.

As an Example the Technique Leading to the Preparation of Tissue Sections from Different Organs of Mice Injected with Gold-tagged Lymphocytes is Described:

Lymphocytes were prepared from mouse spleen as described by U. Schoepf, E. Marecos, R. Melder, R. Jain, and R. Weissleder: “Intracellular magnetic labelling of lymphocytes for in vivo trafficking studies” published in Bio Techniques 1998; 24: 642-651. In short, the mice were sacrificed under anaesthesia and their spleens were removed. Lymphocytes were obtained by being placed in a rotating tissue chopper and diluted with a medium supplemented with 8% fetal bovine serum. After centrifugation, erythrocytes were lysed by resuspending the pellet in 0.83% ammonium chloride in distilled water. One hour later, the solution was transferred to another container leaving the adherent cells in the ammonium chloride jar. This approach results in about 95% lymphocytes as determined by morphology. After centrifugation the pellet was added to 3 ml medium and 1 ml of the AMG-tag-MTS complex, and the vial incubated at 37° C. for 1 h. The suspension was then washed 4 times through a step gradient of 40% Histopaque-1077 (Sigma®) in a Hank's balanced salt solution.

The now gold-tagged lymphocytes were injected into the mice, and 10, 15 and 20 days later, the animals were anaesthetised and perfused transcardially with a 3% glutaraldehyde solution in a 0.1 M phosphate buffer for 10 minutes. The spleen, mesenteric lymph glands, bone marrow and thymus were removed and placed in the fixation solution for at least one hour. Control tissue from mice not injected with AMG-tagged lymphocytes was included.

Blocks of tissue were embedded in paraffin and Epon according to conventional procedures. Tissues that were to be cut in the cryostat, were placed in a 30% solution of sucrose until they sank to the bottom of the vial (1-2 days) and were then frozen in carbon dioxide. Sections from the three sources were placed on glass slides, dipped in a 0.5% gelatine solution and dried.

How to Silver Enhance the AMG-tag-MTS Complex:

The commercially available AMG developers as well as the recommended developers in the literature are mostly worked out from the original lactate developer published in 1981 by G. Danscher: “Histochemical demonstration of heavy metals. A revised version of the sulphide silver method suitable for both light and electron microscopy” in Histochemistry 71:1-16.

Autometallography

The slides were placed in jars and covered with the autometallographic developer. These jars were kept in a water bath at 26° C. in complete darkness. Because the developing time is dependent on the thickness of the sections, it is recommended that different developing times are used on parallel series of sections to avoid overlooking delicate traces of gold. After development, the slides are washed in gently running tapwater at 40° C. for at least 40 min to remove the protecting gelatine and rinsed in distilled water.

The Developer Contains the Following Ingredients

A. Protecting colloid. One kg crystalline gum arabic is dissolved in 2 l deionised water by intermittent stirring. Five days later the solution is filtered through layers of gauze. Suitable portions of colloid can be frozen in plastic jars and stored for at least 6 months.

B. Citrate buffer. Citric acid 1 H₂O, 25.5 g; sodium citrate 2 H₂O, 23.5 g; deionised water 100 ml (pH 3.5).

C. Reducing agent. Hydroquinone, 0.85 g; deionised water, 15 ml.

D. Silver ion supply. Silver lactate, 0.11 g; deionised water, 15 ml. Silver lactate is highly sensitive to light, and the solution should be carefully protected from light.

Preparation of the Developer

Solution A 60 ml; solution B 10 ml; solution C 15 ml; solution D 15 ml. Solutions A, B, and C are carefully mixed, and solution D is added immediately before use.

Post-Staining Treatment of Frozen and Paraffin Embedded Sections

This step implies counterstaining with thionin, neutral red, hematoxylin-eosin, or any other suitable staining solution. After being rinsed, they are dehydrated, cleared and cover-slipped in Dammar resin.

Treatment of Epon Sections

The Epon sections can be counterstained with toluidin blue (1%) After light microscopic analysis a section to be processed for electron microscopy is covered with a drop of Epon solution and a blank Epon block is placed over it. The resin is allowed to polymerise and the section is removed from the glass slide after being quickly heated on a hot plate. The ultrathin sections, made in conventional ways, can be stained with uranyl and lead.

Because of an extremely low unspecific staining, the Epon-embedded sections or metachrylate embedded sections are superior to both paraffin-embedded and frozen sections for the light microscopical localisation of gold.

Further Applications

The possibility of tagging cell organelles opens up a complete new line of research, as cell organelles, for example nuclei loaded by MTS tagged colloidal gold can be removed from its original cell and implanted into another cell including early stages of blastula or striated muscle cells. If studying the progeny of injected cells in an organism, the functional potentials and number of divisions can then be monitored based on the amount of AMG-tags in the cells. Comparably, it will be possible to follow mitochondria and other cell organelles inoculated into a receptor cell.

The present invention allows tracing of cells/bacteria/mitochondria and their progeny in organisms and cultures. For instance, it is feasible to AMG-tag sperm cells from two different males with differently sized colloidal gold particles with MTS. The two ejaculates then be mixed and used for insemination for later analysis of possible differences in the capability of sperms from the two males that reach the ampulla of tuba uterine, and the fertilising sperm cell identified as the male pronucleus will be AMG-tagged with either of the two seizes of gold particles. Also, a fraction of sperm cells from a male can be isolated and treated in different ways, AMG-tagged, and later traced, in order to evaluate their capacity.

Likewise, isolated stem cells origin from, for example, embryos or umbilical cords can likewise be tagged and traced.

Cancer cells can be AMG-tagged and after injection into the experimental animals, it is possible to observe which organs/tissues are invaded by the cells and where they result in tumours.

Also, biopsies from a bone marrow donor can be tagged and injected into the recipient—later biopsies will show the location and dynamics of the injected cells.

The invention makes it possible to follow the fate of transplanted cells and their progenitor cells for 15-20 generations. It allows studies of the behaviour of isolated cells or tissues, for example exposed to a certain drug or toxic molecule. The set-up enables studies after homotransplantation or xenotransplantation and is valuable for embryological studies and when establishing the origin and turnover of cells like macrophages and osteoclasts.

Also, the MTS-gold-AMG tags can be used to trace axonal connections in the central nervous system. After local injection of the probes with AMG-tags into the brain, all the cells and axons that are exposed to the tags will be loaded, and the gold particles spread into every part of the cell. In the case of neurons, dendrites and axons will be filled with tags. This line of events leads to a detailed picture of the neurons and their ramifications that are connected to that particular location.

Different sized colloidal cores will after AMG development maintain their relative differences in size. This implies possibilities of identifying cells marked with different sized gold particles. It is thus possible to follow different cell lines of the same type of cells, for example taken from different persons, and differentiate them in the receptor organism.

As the gold particles remain in organic entities, also after division, the progeny of cells, bacteria, or fungi can be traced, as described above. Counting the number of AMG-tags in entities reveals the number of generations that has led to the actual entity after multiple division of the cell, bacteria or fungi. According to the aforementioned article by Josephson et al., 10⁷ magnetic iron oxide particles could be loaded per cell. For organometallic probes according to the invention, a likewise number is easily possible, which means that at least 15-20 generations can be traced.

Tracing of micro-organisms like bacteria and protozoa is another field of application. In this case, inoculation of AMG-tagged bacteria or protozoa in cultures or in organisms can give valuable insight.

The technique can also be used to irradiate certain tissues, where a particular kind of cells is known to accumulate, by loading the isolated AMG-tagged cells with a radioactive isotope.

To the MTS-gold colloid conjugates can further be attached molecules that selectively bind to receptors, particular for a certain group of cells in an organism or in a culture, or the like, causing uptake of gold in these cells, but not in other cells. This implies the possibility of marking certain types of cells by intra venous injection of the special marked MTS-gold in the living organism. Thereafter, the now marked cells can be followed for a high number of generations by way of analyzing biopsies

The technique according to the invention is also suited for loading liposomes with colloidal gold particles for easy monitoring in tissues.

A severe question for any organ transplantation is the acceptance of the cells of the organ to be transplanted. If test cells from the donor are marked, for example bone marrow cells, they may be injected into the recipient and traced in biopsies as an extra evaluation of the acceptance of the donor cells by the recipient prior to transplantation.

As it clearly appears from the foregoing, an organometallic probe according to the invention has a broad range of application. Technological progress in the AMG-tag field can be expected on two fronts for further development of the invention, namely development of new efficient AMG-tags, and development of new and perhaps even more efficient MTS moieties.

1. An organometallic probe comprising a colloidal core with a membrane translocating signal (MTS) moiety attached thereto, wherein said core is a gold particle.

2. An organometallic probe according to claim 1, wherein said gold particle is directly bound to said MTS.

3. An organometallic probe according to claim 1, wherein said MTS is an HIV-tat fragment, a herpes virus protein, a MTS peptide derived from anti-DNA monoclonal antibody, or the third helix of the homeodomain of Antennapedia.

4. An organometallic probe comprising a colloidal core with a membrane translocating signal (MTS) moiety attached thereto, wherein said core contains at least one—from the group consisting of a metal, a metal selenide or a metal sulphide.

5. An organometallic probe according to claim 4, wherein said metal, metal selenide, or metal sulphide is directly bound to said MTS.

6. An organometallic probe according to claim 4, wherein said core comprises at least one from the group consisting of gold, bismuth sulphide, bismuth selenide, mercury sulphide, mercury selenide, silver sulphide, silver selenide, copper sulphide and copper selenide.

7. An organometallic probe according to claim 4, wherein said MTS is an HIV-tat fragment, a herpes virus protein, a MTS peptide derived from anti-DNA monoclonal antibody, or the third helix of the homeodomain of Antennapedia.

8. A method for tracing a biological entity or a progeny of said entity, said method comprising internalising into said entity an organometallic probe having a colloidal core with a membrane translocating signal (MTS) moiety attached thereto, wherein said core is a gold particle, and wherein said method comprises exposing said entity or a progeny of said entity to a treatment for silver enhancement of said organometallic probe.

9. A method according to claim 8, wherein said MTS is at least one from the group consisting of an HIV-tat fragment, a herpes virus protein, a MTS peptide derived from anti-DNA monoclonal antibody, or the third helix of the homeodomain of Antennapedia.

10. A method according to claim 8, wherein said entity is at least one from the group consisting of a cell, a cell organelle, a mitochondrion, a lysosome, a vesicle, a protozoon, a bacterium, and a fungi.

11. A method for tracing a biological entity or a progeny of said entity, said method comprising internalising into said entity an organometallic probe having a colloidal core with a membrane translocating signal (MTS) moiety attached thereto, wherein said core contains at least one from the group consisting of a metal, a metal selenide, and a metal sulphide, and wherein said method comprises exposing said entity or a progeny of said entity to a treatment for silver enhancement of said organometallic probe.

12. A method according to claim 11, wherein said core comprises at least one from the group consisting of gold, bismuth sulphide bismuth selenide, mercury sulphide, and mercury selenide, silver sulphide, silver selenide, copper sulphide and copper selenide

13. A method according to claim 11, wherein said MTS is at least one from the group consisting of an HIV-tat fragment, a herpes virus protein, a MTS peptide derived from anti-DNA monoclonal antibody, or the third helix of the homeodomain of Antennapedia.

14. A method according to claim 11, wherein said entity is at least one from the group consisting of a cell, a cell organelle, a mitochondrion, a lysosome, a vesicle, a protozoon, a bacterium, and a fungi.

15. A method for identifying a biological entity in a surrounding substance comprising isolating said entity, binding to said entity an organometallic probe having a colloidal core with a membrane translocating signal (MTS) moiety attached thereto, said core containing at least one from the group consisting of a metal, a metal selenide, and metal sulphide, introducing said entity into said substance, exposing at least part of said substance to a treatment for silver-enhancement of said organometallic probe for identification of said organometallic probe by microscopic investigation.

16. A method according to claim 15, wherein said colloidal core comprises at least one from the group consisting of gold, bismuth sulphide bismuth selenide, mercury sulphide, mercury selenide, silver sulphide, silver selenide, copper sulphide and copper selenide.

17. A method according to claim 15, wherein said MTS is at least one from the group consisting of an HIV-tat fragment, a herpes virus protein, a MTS peptide derived from anti-DNA monoclonal antibody, or the third helix of the homeodomain of Antennapedia.

18. A method according to claim 15, wherein said entity is at least one from the group consisting of cell, a cell organelle, a mitochondrion, a lysosome, a vesicle, a protozoon, a bacterium, and a fungi.

19. A method according to claim 15, wherein said substance is an organism, an organ, an embryo, a bacteria culture, or a cell culture.

20. A method according to claim 15, wherein said part of said substance is a biopsy or a smear.

21. A kit containing a suspension with organometallic probes, such probe comprising a gold particle with a membrane translocating signal (MTS) moiety directly bound thereto and a developer agent for silver enhancement of said probe.
